(19) United States
(12) Patent Application Publication
(10) Pub. No.: US 2011/0033922 A1
(43) Pub. Date: Feb. 10, 2011

(54) MICROCHIP-BASED ACOUSTIC TRAPPING OR CAPTURE OF CELLS FOR FORENSIC ANALYSIS AND RELATED METHOD THEREOF

(76) Inventors: James P. Landers, Charlottesville, VA (US); Katie Hall, Charlottesville, VA (US); Johan Nilsson, Barred (SE); Thomas Laurell, Lund (SE); Mikael Evander, Lund (SE)

Correspondence Address: SCHWEGMAN, LUNDBERG & WOESSNER, P.A., P.O. BOX 2938, MINNEAPOLIS, MN, 55402, US

(21) Appl. No.: 12/089,320

(22) PCT Filed: Oct. 4, 2006
(86) PCT No.: PCT/US2006/038943

§ 371 Date: Jun. 30, 2010

Related U.S. Application Data
(60) Provisional application No. 60/723,551, filed on Oct. 4, 2005.
(60) Provisional application No. 60/776,751, filed on Feb. 24, 2006.

(51) Int. Cl. C12N 5/071; C12M 1/00

(52) U.S. Cl. 435/325; 435/283.1

(57) ABSTRACT

The present invention provides a method and apparatus for separating by size a mixture of different size particles using ultrasound. The apparatus contains a microchannel having an acoustic transducer thereon. As a mixture of cells having different sizes flows down the microchannel, the ultrasonic radiation traps cells of desired sizes focused at nodes of a standing pressure wave in the microchannel.

This application claims the priority of U.S. Provisional Patent Application Ser. Nos. 60/723,551, filed Oct. 4, 2005 and 60/776,751, filed Feb. 24, 2006.

### BACKGROUND OF THE INVENTION

The advent of microdevice technology for biochemical and chemical analysis has begun to revolutionize the analytical measurement sciences. While the microchip revolution is rooted in ultrafast separations, recent forays seek to move laborious and time-intensive steps for sample collection, lysis, extraction, and reaction to microchips¹⁻⁵. A number of emerging “lab-on-a-chip” systems have been described to address sample preparation issues, this has been extended to the field of cellomics—the manipulation of cells, and even single cells, in microfluidic devices. Developments in this area will be key to the achieving a more complete micro-total-analysis system (μ-TAS).

Cell manipulation on microdevices has been demonstrated extensively. For example, Ramsey and colleagues demonstrated transport and lysis of cells on microfluidic devices.⁶ Cell lysis, induced chemically or with an electric field, was utilized to release cell lysate for further analysis. Similarly, Harrison et al. developed a microchip-based method for detection of cell lysate including the use of enzymatic reactions.⁷ Cell separations on microdevices have been demonstrated by a number of means, including the use of electric fields or other physical means. Sorting of sub-cellular components has been demonstrated on a microdevice using isoelectric focusing, in which the pH gradient is set up across a channel and the flow (containing electric field have also been described extensively, using electroosmotic flow for fluid flow throughout the device.^{9, 10} Additionally, dielectrophoresis has been shown effective for separation and manipulation of cell and bacteria in microdevices.^{11, 12, Arai, 2001 #56 13} Micro-fluorescence activated cell sorters have been developed for sorting fluorescently-labeled cells in microdevices.¹⁴⁻¹⁶ A microdevice cell separation method by application of an electric field and introduction of a filter, without damage to the cells, has been invented by Yasuda.¹⁷ Filters, in particular, have been utilized extensively for trapping various cell types, which utilize the adhesiveness of white blood cells to further enhance the separation.^{18, 19} A separation based upon differential settling and adsorption of sperm and epithelial cells in microfluidic devices has been demonstrated.²⁰

Particles subjected to acoustic waves are influenced by acoustic radiation forces, which are particularly strong in standing wave fields²¹. The forces can be divided into axial and transverse components of the primary radiation force, and secondary particle-particle interactions due to scattering of incident waves²². The acoustic properties of the particulate material as compared to the surrounding medium determine whether the primary radiation force is directed towards the pressure nodes or antinodes in a standing wave. The magnitude of the radiation force is proportional to the acoustic frequency²² and for particle manipulation it is therefore advantageous to increase the frequency to the ultrasonic region. Consequently ultrasonic has successfully been used to manipulate particles or biological material, e.g. as acoustic tweezers²³ and for particle separation from continuous fluid flow in macro-²⁴ and microscale devices.²⁵ Two-dimensional trapping and manipulation of microorganisms has been performed using orthogonal standing waves.²⁶ Size-selective ultrasonic trapping of microbeads in capillaries has also been investigated in order to allow separation of immunocomplexes for trace-amount protein detection.^{27, 28} Other bio-related applications making use of acoustic forces include separation of fat from blood during cardiovascular surgery²⁹ and the retention of mammalian cells in cell culture fermentations.³⁰ The combination of acoustic trapping and microsystems has been examined for development of bead based bioassays.^{31, 32}

Patents using ultrasonic radiation to separate cells have been shown in U.S. Pat. Nos. 6,332,541 to Coakley et al. (the '541 patent); 6,929,750 to Laurell et al. (the '750 patent); and 7,108,137 to Lal et al. (the '137 patent); the disclosures of which are incorporated herein by reference. The '541 and '750 patents are drawn to cell separation by applying an ultrasonic wave in a direction orthogonal to the direction of flow. This system separates the cells but does not trap it at a particular location within the channel. The '137 patent apply acoustic radiation in a longitudinal direction, and therefore, does aggregate cells at various locations along the flow path rather than at a single define position directly above the transducer.

With an interest in creating a μ-TAS for totally-integrated analysis of DNA evidence from forensic sample, a focused effort has been invested in the development of microminiaturized fluidic device for isolating sperm cells from sexual assault evidence. The benefit of such an invention is that the separation of cells, such as sperm from cell mixtures, in the conventional analysis of sexual assault evidence is time-consuming, labor-intensive and results in relatively poor separation efficiency.³³ Conventional differential extraction (DE)—the separation of male (sperm cell) and female (epithelial cell) DNA is the currently accepted method and is based on that proposed by Gill³⁴ which has been morphed into the various protocols used today in crime laboratories. This method yields separate fractions of male and female DNA, essential to obtaining DNA profiles from both a victim and perpetrator in a forensic sexual assault case. Conventional DE is a chemical process that induces the differential lysis of the cells by exploiting the differences in the stability of the cell membranes on sperm and epithelial cells. This method is initiated by lysing the vaginal epithelial cells under mild conditions that allow the sperm cells to remain intact. The intact sperm cells (predominately the heads, as tails are solubilized under mild lysis conditions) are pelleted by centrifugation, allowing the now released DNA from the epithelial cells to be removed in the supernatant. The pelleted sperm cells are then resuspended and lysed in a buffer that contains dithiothreitol (DTT), a reagent that reduces disulfide bonds on the sperm cell surface, and the DNA is extracted independently. This method typically requires a minimum of 3 hours, and is often allowed to incubate overnight. While this method has been used for a number of years, the presence of female DNA in the sperm cell fraction leads to co-amplification of the female alleles which complicate the genetic fingerprint provided by the male DNA.³³ This creates difficulties with the interpretation of evidence and compromises the effectiveness of connecting the DNA fingerprint to the perpetrator (lowers the probabilities when presenting statistical data) in court proceedings. Aspects of various embodiments of the present invention described herein seek to improve the purity of the male and female fractions obtained, so as to improve forensic DNA analysis of sexual assault evidence.

The use of acousto-trapping for isolation of sperm cells from the biological mixture (sexual assault evidence) significantly reduces analysis times and, perhaps most importantly, increases sample purity. While this technology is described for cell selection/capture in fluidic microdevices known for manipulation of nanoliter-picoliter volumes, the mechanism for sperm cell capture from a cell mixture it selective under conditions that allow for high volumetric flow rate and, hence, milliliter volume samples. This concept is supported by the already demonstrated application of lipid removal from recovered blood during thoracic surgery³⁵ is highly amenable to multiplexing, which allows for analysis of sample sizes ranging from hundreds of microliters to milliliters in minutes. Moreover, with the microscalar fluidic structures used in these devices, multisample capability can easily be added. Greater purity of the sperm cell fraction results in an increased likelihood for identification and prosecution of the perpetrator in sexual assault casework. In addition, greatly enhancing the rate of evidence analysis will diminish the backlog of cases awaiting DNA analysis in many criminal laboratories.

### SUMMARY OF THE INVENTION

An aspect of various embodiments of the present invention is to, but not limited thereto, utilize acoustic standing waves in a fluid-filled microchannel in an analytical microchip device to create a chip-based acoustic differential extraction (ADE) microdevice. This device will allow for the selective isolation of cell, preferably sperm cells, from small or large volumes flowing streams containing the cells and cellular material obtained, for example from forensic evidence.

The advantages of the current system include: 1) the rapid manner in which cells can be trapped in the near field of the ultrasonic transducer, 2) its ease of use, 3) the effectiveness for isolating a pure cell fraction from evidentiary material in forensic samples, 4) the effectiveness relative to separation of sperm cells from other biological material in sexual assault evidence by conventional means, 5) the ability to separate free DNA from a cellular mixture, 6) its versatility in handling microliter or milliliter scale samples (hence, large volume reduction), 7) its tenability for selective cell capture, 8) the concentrating effect that stems from its ability to capture cells from large volumes (milliliters) and release them in extremely small volumes (microliters-nanoliters), 9) amenability to multi-sample analysis (multiplexing), 10) the ability of the microchip to prevent contamination of evidentiary material, 11) the ability define configuration wherein the transducer is part of a platform and not part of the chip and 12) the low cost and disposability of the chip.

The present invention provides a method and apparatus for separating by size a mixture of different size particles using ultrasound. The apparatus contains a microchannel having an acoustic transducer thereon. As a mixture of cells having different sizes flows down the microchannel, the ultrasonic radiation, applied in a direction perpendicular to the flow, traps cells focused at nodes of a standing pressure wave in the microchannel, directly above the transducer. The size selection of the cell to be trapped is based on the trapping force of the ultrasonic radiation which can be tuned to trap the desired cell size.

Further objects, features and advantages of the invention will be apparent from the following detailed description when taken in conjunction with the accompanying drawings.

### BRIEF SUMMARY OF THE DRAWINGS

FIG. 1 is a drawing of a longitudinal section of an embodiment of the present invention having of two cell trapping sites.

FIG. 2 is a drawing showing the schematic of a multilayer transducer.

FIG. 3 is a schematic illustration of an acoustic differential extraction device design.

FIG. 4 is a photomicrograph depiction of sperm cell trapping above the ultrasonic transducer element in the ADE microdevice

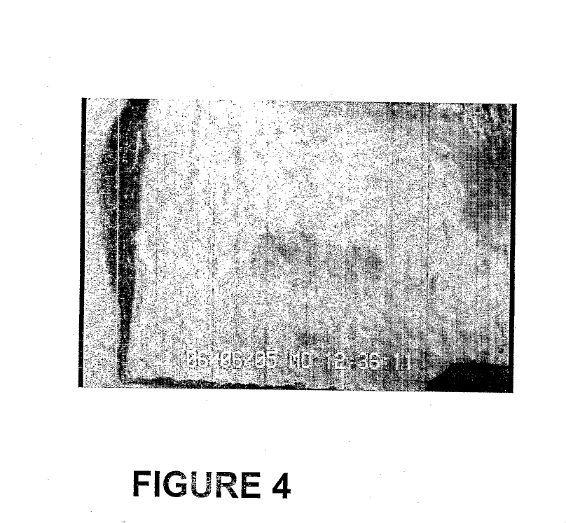

FIG. 5 is a photomicrograph depiction of bacteria and other non-sperm material collected in the antinodes of the ultrasonic wave from a forensic sample.

FIG. 6 is a cross-sectional drawing of the cell trapping site showing a standing pressure wave.

FIG. 7 is a drawing of a longitudinal section of an embodiment

### DETAILED DESCRIPTION OF THE INVENTION

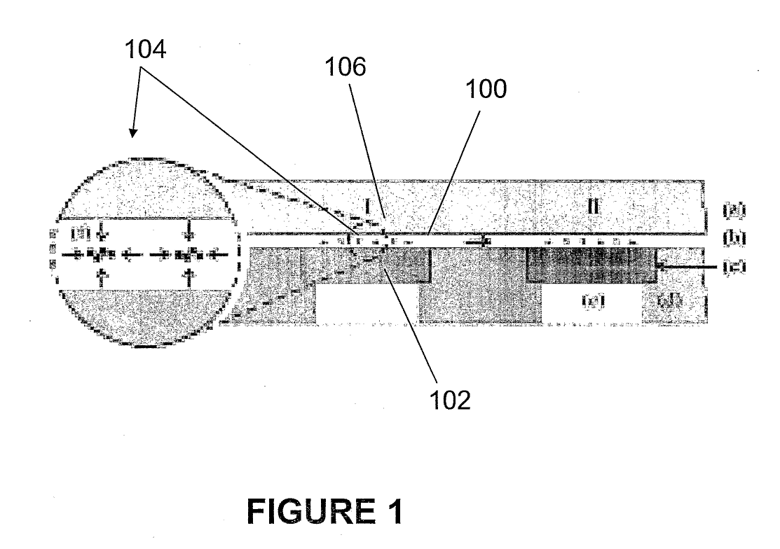

FIG. 1 shows a preferred embodiment of the present invention. The apparatus contains a microchannel 100 having at least one cell trapping region 104. The cell trapping region 104 contains an acoustic transducer 102, preferably at the bottom of the microchannel as shown, and a reflector 106, preferably a glass reflector. In certain embodiments, multiple cell trapping regions are located along the flow path of the microchannel 100, where each flow path aggregates a different cell size.

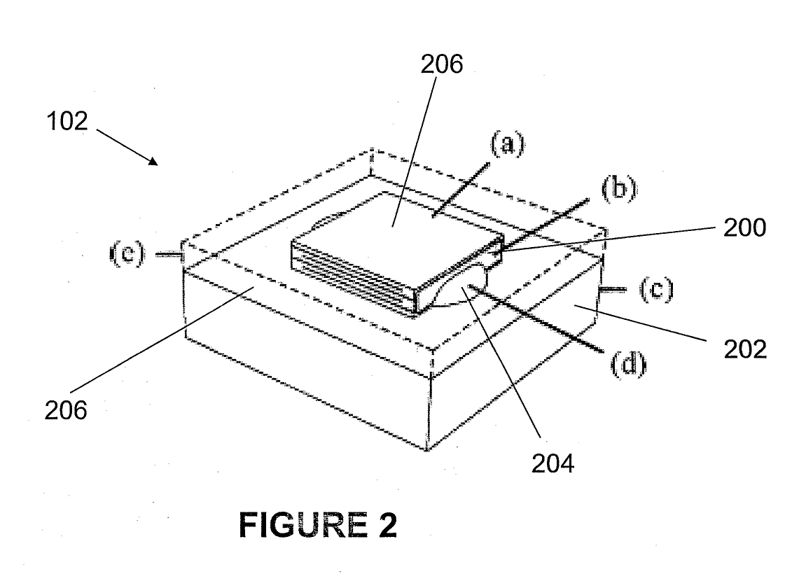

In an embodiment, the ultrasonic transducer 102 is fabricated using a screen-printed PZT-multilayer device. The detailed description of the actuator fabrication and means of contacting is given in Lilliehorn et al.³¹, which is incorporated herein by reference. FIG. 2 shows a schematic of a multilayer ultrasonic transducer 102 containing a transducer element 206 with an external silver electrode 200 connected to the circuitry on a printed circuit board 202 with conductive silver epoxy 204. The board 202 is covered with epoxy 206. Other ultrasonic transducers may also be appropriate for the present invention, including those described in the '750, '541, or '137 patent.

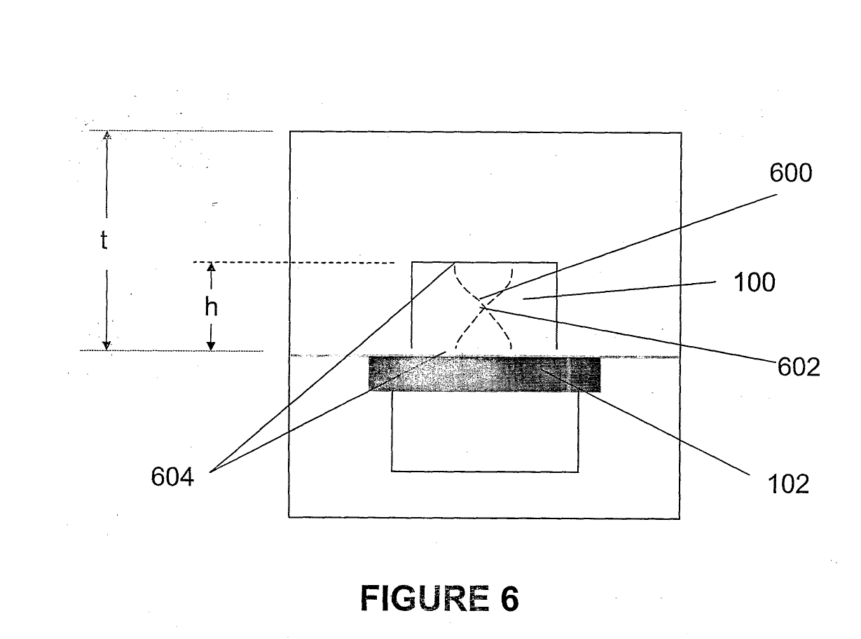

During use a cell mixture, preferably in a fluid media, flows into the microchannel 100 using, for example a pump. An acoustic radiation is applied the direction of the axis of the microchannel 100 generating a standing pressure wave 600 (see FIG. 6). The standing pressure wave 600 contains a node 602 and antinodes 604 that trap the desired cell particles. In a most preferred embodiment, the thickness t of material above the channel is an odd number of ¼ wave length [(2n+1)λ, where n is a whole number], and the height h of the channel is ½ wavelength (λ/2). The selectivity of the system may be tuned a described below.

The standing wave is set by the distance between the transducer surface and the reflector surface, which defines the fundamental acoustic resonance mode of a half wavelength standing wave in the microchannel. In the acoustic trapping device for the present invention the half wavelength distance is preferably approximately 61 μm, which corresponds to a 12.4 MHz fundamental resonance criterion.

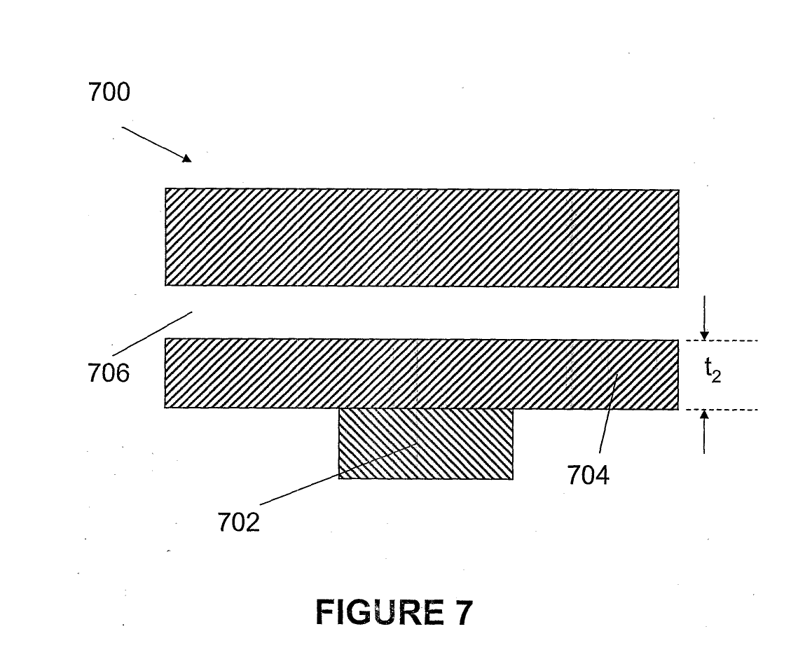

In one embodiment, the microchip can be designed such that the transducer element is part of a separate platform that does not come into fluidic contact with the forensic sample. In this embodiment, when trapping cells, the microchip substrate (e.g. glass) would be positioned in contact (either permanently or temporarily) with the transducer element, thus decreasing the chances of sample contamination, while making the microchip more cost-effective and, perhaps, disposable. In this case, the microchip is separate from the transducer and does not form the bottom of the microchannel as illustrated in FIG. 1. As such, the chip, having the microchannel therein, does not have to be fabricated with an attached and expensive transducer. It is important to properly design the microfluidic chip so that when it is placed on top of a transducer, acoustic radiation can be delivered into the microchannel through a thin glass. FIG. 7 illustrates this embodiment where the microfluidic chip 700 sits on top of a transducer 702, where a bottom layer 704 of the chip 700 separates the microchannel 706 from the transducer 702. For proper function and delivery of the acoustic radiation to the channel, it is critical that the thickness t₂ of the bottom layer 704 should be odd number of ¼ wavelength [(2n+1)λ, where n is a whole number]. In this embodiment, the microfluidic chip is not physically attached to the transducer, but is only placed on top of the transducer when it is in operation.

The dimension of the channel (transducer to reflector distance) defines the fundamental resonance of the resonator. The acoustic trapping force is directly proportional to the standing wave frequency and thus with a reduced distance between the transducer and the reflector the higher the fundamental resonance frequency will be and consequently a higher acoustic trapping force is obtained. The width of the microchannel, a priori, is not a limiting factor and, thus, if a higher capacity is needed more material can be trapped by a wider transducer. On the other hand, channels that are too wide may eventually compromise the benefits of a microfluidic format.

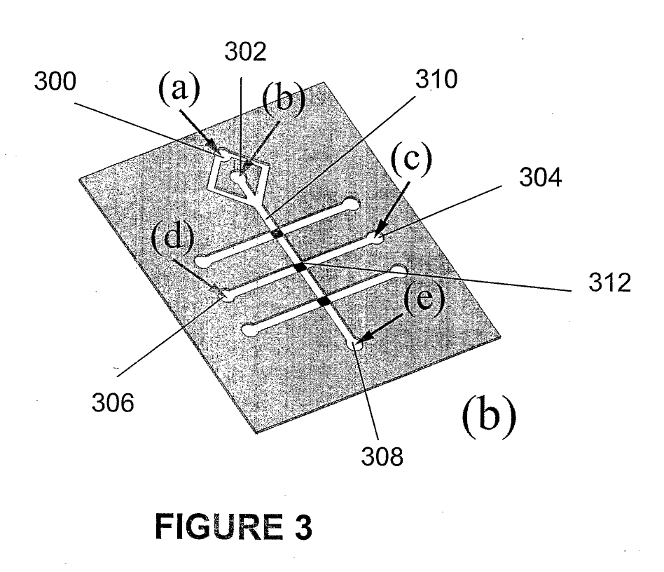

Preliminary experiments show that the cells are initially clustered in a monolayer. Others have reported the same observation in macroscopic acoustic traps.³⁶ When operating the device, the particles and/or cells are collected in a single layer, enclosing several hundred or even thousands of particles/cells (of course depending of the spatial size of the trapping region). As the trap becomes overloaded, multiple layers and aggregates are formed.³⁷ This is significant as it pertains to the effectiveness of this method for trapping cells from forensic or biological samples. The predominately planar accumulation of cells decreases the potential for contamination of the collected cells with other biomolecules. For example, as it pertains to the isolation of sperm cells from sexual assault evidence, any free DNA from lysed female cells (e.g., epithelial cells or white blood cells) is less likely to be nonspecifically trapped in a planar, monolayer-like grouping of cells than would be expected with a three-dimensional cluster of cells (where much opportunity would exist for trapping of free DNA. Moreover, the planar collection of cells can be washed extensively with whatever reagents are desired in order to diminish any trapping of free DNA. This latter embodiment describes the acousto-differential extraction (ADE) device. A generalized description of the apparatus used is seen in FIG. 3. In this device, the buffer is introduced to the main channel 310 through the buffer inlet 300 and sample introduced at the sample inlet 302. Cells are trapped in the trapping region 312. Trapped cells are collected by initiating flow from side channel inlet 304 to side channel outlet 306, where trapped material is collected for further sample processing. The untrapped material is collected at the main channel outlet 308.

One embodiment of a method for ADE involves the step of the conventional DE prior to injecting the sample into the microchannel. Vaginal epithelial cells would be selectively lysed (e.g., by the procedure described by Gill et al.³⁴) and, thus, the sperm cells trapped from a biological mixture containing epithelial cell lysate. Sperm cells (and other particulate matter) are trapped in the standing wave of the ultrasonic transducer, while DNA from the lysed cells is not trapped, but carried with the fluid flow in the channel. Once the epithelial cells are lysed, according using the Gill buffer or other means, sample is flowed (using a syringe pump or other means depending upon sample volume) into the microchannel, where flow is directed over the transducer(s).

A second embodiment of a method for ADE does not require that the cells be lysed but, instead, separates them from the sperm cells intact by trapping at a second transducer. In this embodiment, various cell types could be trapped by a series of transducers. The force acting upon the particle, as described in equation 1, illustrates the utility of the method for trapping particles of various physical properties in the various standing waves.

$$\begin{matrix}{F}_{r} = - ( \frac{π {P}_{0}^{2} {V}_{c} {β}_{w}}{2 λ} ) \cdot Φ ( β , ρ ) \cdot sin ( \frac{4 π z}{λ} ) & ( 1 ) \\ Φ = ( 5 {ρ}_{c} - 2 {ρ}_{w} ) / ( 2 {ρ}_{c} + {ρ}_{w} ) - ( {β}_{c} / {β}_{w} ) & ( 2 )\end{matrix}$$

Where P₀=the applied acoustic pressure amplitude
V_{c}=particle volume
β_{w}=compressibility of the liquid
β_{c}=compressibility of the particle
λ=wave length of the sound wave
z=particle distance to the node
δ_{c}=density of the particle
δ_{W}=density of the liquid
Φ(β, ρ)=the acoustic contrast factor
Fᵣ=the acoustic radiation force

As previously described, the trapping force is dependent of the distance between the transducer surface and the reflector a smaller distance yields a higher trapping force. This is a fundamental approach to control the trapping efficiency (a smaller channel height results in a higher resonance frequency and thus a better trapping force). The force is also highly dependent of the size of the particle to be trapped and is, for each cell-type, essentially a fixed parameter. The next factor in equation 1 to take into account is the Φ-factor (commonly referred to as the ‘acoustic contrast factor’), which is defined by the densities of the carrier fluid, the particle and the ratio of the compressibility's between the carrier fluid and the particle (equation 2). The parameters to modulate, from an engineering perspective, involve defining the carrier fluid with respect to compressibility and density. In ultracentrifugation work, the carrier media is selected with respect to suitable density. Likewise, in the acoustic trapping experiments, selection can be made in a similar manner with respect to fluid density, but now, fluid compressibility is an additional parameter that can be used optimize the trapping capability of the system. Another alternative is to use the much stronger forces acting on the larger cells (e.g., epithelial cells) to induce a selective trapping. This could be achieved by finding the threshold where the magnitude of the acoustic forces are strong enough to trap epithelial cells but don't effect smaller cells (e.g., sperm cells). Consequently, as it pertains to the separation of epithelial cells from sperm cells, epithelial cells would be trapped in the standing wave generated by one transducer, while sperm cells are trapped in the standing wave generated by a second transducer in a spatially-distinct part of the microfluidic architecture. Such selectivity can be obtained by tuning the amplitude output of the waveform generator with the physical properties of the cell types.

Another embodiment of this method involves the trapping of cells, as described earlier, and release of cells for further processing on the microdevice, including, but not limited to, cell lysis and DNA extraction. In this embodiment, the cell trap of the present invention can be used with other existing microfluidic apparatus including those disclosed in U.S. Patent Application Publication Nos. 2006/0084185, 20050287661, 20040131504, all to Landers et al. and are incorporated herein by reference.

Other than the cell trapping site, microfluidic devices may also include micromachined fluid networks. Fluid samples and reagents are brought into the device through entry ports and transported through channels to a reaction chamber, such as a thermally controlled reactor where mixing and reactions (e.g., synthesis, labeling, energy-producing reactions, assays, separations, or biochemical reactions) occur. The biochemical products may then be moved, for example, to an analysis module, where data is collected by a detector and transmitted to a recording instrument. The fluidic and electronic components are preferably designed to be fully compatible in function and construction with the reactions and reagents.

There are many formats, materials, and size scales for constructing microfluidic devices. Common microfluidic devices are disclosed in U.S. Pat. Nos. 6,692,700 to Handique et al.; 6,919,046 to O'Connor et al.; 6,551,841 to Wilding et al.; 6,630,353 to Parce et al.; 6,620,625 to Wolk et al.; and 6,517,234 to Kopf-Sill et al.; the disclosures of which are incorporated herein by reference. Typically, a microfluidic device is made up of two or more substrates or layers that are bonded together. Microscale components for processing fluids are disposed on a surface of one or more of the substrates. These microscale components include, but are not limited to, reaction chambers, electrophoresis modules, microchannels, fluid reservoirs, detectors, valves, or mixers. When the substrates are bonded together, the microscale components are enclosed and sandwiched between the substrates.

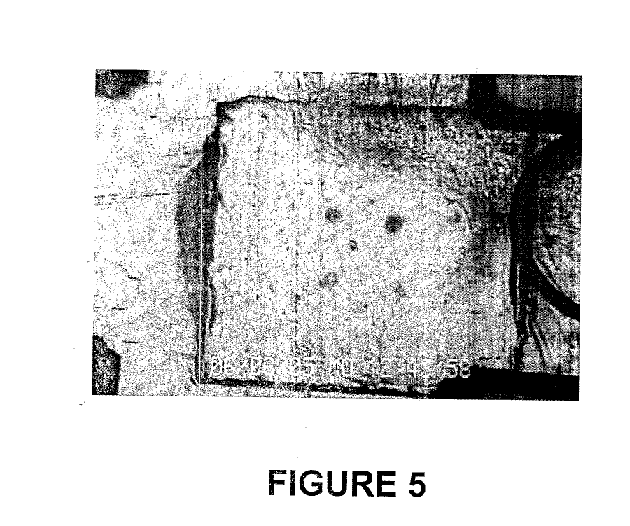

Other cells of forensic importance (and often encountered in evidentiary material) include microorganisms. In another embodiment of the method described, these cells may also be isolated by trapping or selectively not trapping these cells. For example, FIG. 5 shows the trapping of bacteria from a mock sexual assault sample in the antinode of the transducer.

A number of designs can be envisioned for the ADE chip and, accordingly, there are a number of different approaches that could effectively lead to recovery of the trapped sperm cells can be from the forensic sample.

A potential protocol for assembling an ADE microdevice, as represented by a glass microfluidic chip bonded to the transducer chip, is as follows:
1) A glass chip is fabricated to have a channel depth that corresponds to half a wavelength of the desired working frequency of the ADE (at current working frequency of 12.4 MHz that is 61 μm). The configuration of the microchannel above the transducer does not need to be straight walled, and can have the U-shaped channels commonly found in etched glass devices. The reflective surface above the transducer needs however to be planar to ensure a good reflected wave.
2) The transducer chip, fabricated by the method previously reported³¹ is bonded to the chip by the use of a hydrogel as an adhesive. The chip contains the transducers and the electrical wiring to actuate the transducers at the desired frequency.
3) One approach to ensure a tight fit the transducer chip and the glass channels is to hold them together with a brass fixture. However, this would not be needed if any one of a number of bonding processes were carried out to adhere the transducer chip to the glass.
4) Valves can be incorporated into the microfluidic architecture to control the flow of solutions and cells through specific, predefined fluidic paths for spatial separation and capture of cell and fluid fractions. There are a number of different valving approaches that could be used for this including physical valving,^{38,39} electrokinetic valving,⁴⁰ passive valving as detailed in Duffy et al.,⁴¹ and passive flow control with fluidic diodes, capacitors, inductors and band pass filters.

A method trapping sperm cells from a biological sample with an ADE microdevice, as represented by a transducer bonded to, e.g., a glass microfluidic chip, is as follows:
1) Cells obtained from forensic evidence (examples include but are not limited to vaginal swabs and bedsheets) in an elution buffer (i.e., phosphate buffered saline, Gill buffer, or other liquid) are perfused into the microdevice channels using a syringe pump or other pumping means.
2) The sperm cells are trapped in the standing wave of the transducer.
3) If desired, the trapped sperm cells can be washed by infusing buffer or water through the microchannel.
4) After the desired cells are trapped, flow in the cross-channel can be initiated, the standing wave turned off, and the cells released. The flow in the cross-channel directs the released cells into the outlet of interest, for collection or further manipulation on-chip. This collection of the trapped materials can be completed with or without on-chip valving to aid in sample collection.
5) The non-trapped cells can be collected from the outlet reservoir throughout the perfusion of sample and sample washing. This can be accomplished by various means, including but not limited to attaching tubing to the outlet reservoir and collecting the flow-through in an attached receptacle.

The removal of cells, materials, analytes, etc., from these devices should be appreciated by and are with in the capability of those skilled in the art.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

### LITERATURE CITED

The following references are hereby incorporated by reference herein in their entirety:
(1) Woolley, A. T.; Hadley, D.; Landre, P.; deMello, A. J.; Mathies, R. A.; Northrup, M. A. Anal. Chem. 1996, 68, 4081-4086.
(2) Gottschlich, N.; Culbertson, C. T.; McKnight, T. E.; Jacobson, S. C.; Ramsey, J. M. J. Chromatogr., B: Biomed. Sci. Appl. 2000, 745, 243-249.
(3) Huhmer, A. F.; Landers, J. P. Anal Chem 2000, 72, 5507-5512.
(4) Breadmore, M. C.; Wolfe, K. A.; Arcibal, I. G.; Leung, W. K.; Dickson, D.; Giordano, B. C.; Power, M. E.; Ferrance, J. P.; Feldman, S. H.; Norris, P. M.; Landers, J. P. Anal Chem 2003, 75, 1880-1886.
(5) Broyles, B. S.; Jacobson, S. C.; Ramsey, J. M. Anal Chem 2003, 75, 2761-2767.
(6) Culbertson, C. T. J., Stephen C.; McClain, Maxine A.; Ramsey, J. Michael.; USA, 2003.
(7) Harrison, D. J. A., Per E.; Arriaga, Edgar; Ocvirk, Gregor; The Governors of the University of Alberta, Can.: USA, 2003.
(8) Lu, H.; Gaudet, S.; Schmidt, M. A.; Jensen, K. F. Analytical Chemistry 2004, 76, 5705-5712.
(9) Li, P. C. H.; Harrison, D. J. Analytical Chemistry 1997, 69, 1564-1568.
(10) Manz, A.; Effenhauser, C. S.; Burggraf, N.; Harrison, D. J.; Seiler, K.; Fluri, K. Journal of Micromechanics and Microengineering 1994, 4, 257-265.
(11) Wang, X. B.; Yang, J.; Huang, Y.; Vykoukal, J.; Becker, F. F.; Gascoyne, P. R. Anal Chem 2000, 72, 832-839.
(12) Huang, Y.; Ewalt, K. L.; Tirado, M.; Haigis, T. R.; Forster, A.; Ackley, D.; Heller, M. J.; O'Connell, J. P.; Krihak, M. Analytical Chemistry 2001, 73, 1549-1559.
(13) Arai, F.; Ichikawa, A.; Ogawa, M.; Fukuda, T.; Horio, K.; Itoigawa, K. Electrophoresis 2001, 22, 283-288.
(14) Fu, A. Y.; Chou, H. P.; Spence, C.; Arnold, F. H.; Quake, S. R. Analytical Chemistry 2002, 74, 2451-2457.
(15) Fu, A. Y.; Spence, C.; Scherer, A.; Arnold, F. H.; Quake, S. R. Nature Biotechnology 1999, 17, 1109-1111.
(16) Weigl, B. H.; Bardell, R. L.; Battrell, C. F.; Hayenga, J.; Lancaster, C.; Graham, P. Clinical Chemistry 2003, 49, A153-A153.
(17) Yasuda, K. T., Kazunori; Japan Science and Technology Agency, Japan: USA, 2004.
(18) Wilding, P.; Kricka, L. J.; Cheng, J.; Hvichia, G.; Shoffner, M. A.; Fortina, P. Anal Biochem 1998, 257, 95-100.
(19) Carlson, R.; Gabel, C. V.; Chan, S. S.; Austin, R. H.; Brody, J. P.; Winkleman, J. Molecular Biology of the Cell 1997, 8, 2407-2407.
(20) Horsman, K. M.; Barker, S. L.; Ferrance, J. P.; Forrest, K. A.; Koen, K. A.; Landers, J. P. Anal Chem 2005, 77, 742-749.
(21) Groschl, M. Acustica 1998, 84, 432-447.
(22) Woodside, S. M.; Bowen, B. D.; Piret, J. M. Aiche Journal 1997, 43, 1727-1736.
(23) Wu, J. R. Journal of the Acoustical Society of America 1991, 89, 2140-2143.
(24) Hawkes, J. J.; Coakley, W. T.; Groschl, M.; Benes, E.; Armstrong, S.; Tasker, P. J.; Nowotny, H. Journal of the Acoustical Society of America 2002, 111, 1259-1266.
(25) Nilsson, A.; Petersson, F.; Jonsson, H.; Laurell, T. Lab on a Chip 2004, 4, 131-135.
(26) Saito, M.; Kitamura, N.; Terauchi, M. Journal of Applied Physics 2002, 92, 7581-7586.
(27) Wiklund, M.; Nilsson, S.; Hertz, H. M. Journal of Applied Physics 2001, 90, 421-426.
(28) Wiklund, M.; Spegel, P.; Nilsson, S.; Hertz, H. M. Ultrasonics 2003, 41, 329-333.
(29) Nilsson, A.; Petersson, F.; Persson, H. W.; Joensson, H.; Laurell, T. Micro Total Analysis Systems 2002, Proceedings of the mTAS 2002 Symposium, 6th, Nara, Japan, Nov. 3-7, 2002 2002, 2, 625-626.
(30) Groschl, H.; Burger, W.; Handl, B. Acustica 1998, 84, 815-822.
(31) Lilliehorn, T.; Simu, U.; Nilsson, M.; Almqvist, M.; Stepinski, T.; Laurell, T.; Nilsson, J.; Johansson, S. Ultrasonics 2005, 43, 293-303.
(32) Lilliehorn, T.; Nilsson, M.; Simu, U.; Johansson, S.; Almqvist, M.; Nilsson, J.; Laurell, T. Sensors and Actuators B-Chemical 2005, 106, 851-858.
(33) Korf, B. R. In Current protocols in Human Genetics; John Wiley & Sons Inc.; New York, N.Y., 1999.
(34) Gill, P.; Jeffreys, A. J.; Werrett, D. J. Nature 1985, 318, 577-579.
(35) Jonsson, H.; Holm, C.; Nilsson, A.; Petersson, F.; Johnsson, P.; Laurell, T. Annals of Thoracic Surgery 2004, 78, 1572-1578.
(36) Coakley, W. T.; Bazou, D.; Morgan, J.; Foster, G. A.; Archer, C. W.; Powell, K.; Borthwick, K. A. J.; Twomey, C.; Bishop, J. Colloids and Surfaces B-Biointerfaces 2004, 34, 221-230.
(37) Nilsson, M.; Johansson, L.; Lilliehorn, T.; Lindvall, M.; Pikur, J.; Almqvuist, M.; Johansson, S.; Laurell, T.; Nilsson, J. Micro Total Analysis Systems 2005, Proceedings of the mTAS 2005 Symposium, 9th, Boston, Mass. 2005, 5.
(38) Studer, V.; Hang, G.; Pandolfi, A.; Ortiz, M.; Anderson, W. F.; Quake, S. R. Journal of Applied Physics 2004, 95, 393-398.
(39) Grover, W. H.; Skelley, A. M.; Liu, C. N.; Lagally, E. T.; Mathies, R. A. Sensors and Actuators B-Chemical 2003, 89, 315-323.
(40) Jacobson, S. C.; Ermakov, S. V.; Ramsey, J. M. Analytical Chemistry 1999, 71, 3273-3276.
(41) Duffy, D. C.; Gillis, H. L.; Lin, J.; Sheppard, N. F.; Kellogg, G. J. Analytical Chemistry 1999, 71, 4669-4678.

What is claimed is:

1. An apparatus for cell separation comprising a microchannel having a cell trapping site, wherein said cell trapping site comprises an acoustic transducer on an opposite side of the microchannel and a reflector surface on another side of the microchannel.

2. The apparatus of claim 1, wherein the acoustic transducer is screen printed PZT-multilayer device.

3. The apparatus of claim 1, wherein the distance between the acoustic transducer and the reflector surface is about 61 μm.

4. The apparatus of claim 1, further comprising a second cell trapping spatially separated from the cell trapping site, wherein said second cell trapping site comprises a second acoustic transducer on an opposite side of the microchannel and a reflector surface on another side of the microchannel.

5. The apparatus of claim 4, wherein said second cell trapping site is down stream of the cell trapping site.

6. The apparatus of claim 4, wherein the second acoustic transducer operates at a different frequency than the first ultrasonic transducer.

7. The apparatus of claim 4, wherein the channel at the second acoustic transducer has a different dimension than the channel at the first acoustic transducer.

8. The apparatus of claim 4, wherein the channel at the second acoustic transducer has a height than the channel at the first acoustic transducer.

9. The apparatus of claim 4, wherein the channel at distances between the transducer and the reflector surface are different at the second acoustic transducer and the first acoustic transducer.

10. The apparatus of claim 1, wherein the transducer is separated from the channel by a layer of material.

11. The apparatus of claim 10, wherein the material is glass.

12. The apparatus of claim 10, wherein the thickness of the material is an odd number of ¼ wavelength of the acoustic radiation generated by the transducer.

13. A method for separating cells comprising the steps of
providing the apparatus of claim 1;
flowing a cell mixture into the microchannel;
activating the ultrasonic transducer at a predetermined frequency.

14. The method of claim 13, wherein the ultrasonic transducer is screen printed PZT-multilayer device.

15. The method of claim 13, wherein the distance between the ultrasonic transducer and the reflector surface is about 61 μm.

16. The method of claim 13, further comprising a second cell trapping spatially separated from the cell trapping site, wherein said second cell trapping site comprises a second ultrasonic transducer on an opposite side of the microchannel and a reflector surface on another side of the microchannel.

17. The method of claim 16, wherein said second cell trapping site is down stream of the cell trapping site.

18. The method of claim 13, wherein the cells being trapped at the cell trapping site are sperm cells.

19. The method of claim 16, wherein the second ultrasonic transducer operates at a different frequency than the ultrasonic transducer.

20. The method of claim 16, wherein the channel at the second ultrasonic transducer has a different dimension than the channel at the first ultrasonic transducer.

21. The method of claim 16, wherein the channel at the second ultrasonic transducer has a height than the channel at the first ultrasonic transducer.

22. The method of claim 16, wherein the channel at distances between the transducer and the reflector surface are different at the second ultrasonic transducer and the first ultrasonic transducer.

23. The method of claim 13, wherein the transducer is separated from the channel by a layer of material.

24. The method of claim 23, wherein the material is glass.

25. The method of claim 23, wherein the thickness of the material is an odd number of ¼ wavelength of the acoustic radiation generated by the transducer.
